(19) United States
(12) Patent Application Publication
(10) Pub. No.: US 2012/0052105 A1
(43) Pub. Date: Mar. 1, 2012

(54) NANOSTRUCTURAL COMPOSITION OF BIOCIDE AND PROCESS OF OBTAINING NANOSTRUCTURAL BIOCIDE NANOCOMPOSITION

(76) Inventors: Anatoly Ivanovich Grigoriev, Moscow (RU); Oleg Igorevich Orlov, Moscow (RU); Umberto Orazio Giuseppe Maugeri, Calvignano (IT); Viacheslav Ivanovich Beklemyshev, Moscows (RU); Igor Ivanovich Makhonin, Moskow District (RU); Ara Arshavirovich Abramyan, Moscow (RU); Vladimir Aleksandrovich Solodovnikov, Moscow (RU)

(21) Appl. No.: 13/138,983

(22) PCT Filed: May 13, 2010
(86) PCT No.: PCT/EP2010/056636

§ 371 Date: Nov. 9, 2011

(30) Foreign Application Priority Data
May 13, 2009 (RU) 2009117737

(51) Int. Cl. A01N 25/00; A01N 59/16; A01P 1/00; A01P 3/00; A61K 33/38; A61K 9/14; A61P 17/00; A61P 17/02; A61P 29/00; A01N 59/20; A61K 33/34; B82Y 5/00

(52) U.S. Cl. 424/405; 424/630; 424/618; 977/773; 977/915; 977/902

(57) ABSTRACT

This invention concerns biocides possessing fungicidal and bactericidal properties which can be used in construction, medicine and other various areas of technics. A nanostructural composition of biocide is realized from nanoparticles of bentonite powders intercalated by ions of Zn2+ and ions of Ag+ and/or ions of Cu2+. The biocides according to the invention are prepared starting from bentonite poweder which is preliminarly enriched with cations of Na+, then treated with 10-20% solutions of inorganic salts of Zn (preferably zinc chloride or zinc sulfate ZnSO4), and from bentonite powders preliminarly enriched with cations of Na+ and then treated with 10-20% solutions of inorganic salts of at least one ion selected in the group consisting of Ag+ ions (preferably silver nitrate) and Cu2++ ions (preferably copper sulfate). The powders of bentonite, intercalated with the Zn2+, Ag+ and/or Cu2+ ions, are .cleaned from acid anions and Na+ salts, and dispersed into nanoparticles mainly of no more than 70 nm.

### FIELD OF THE INVENTION

This invention concerns biocides possessing fungicidal and bactericidal properties which can be used in construction, medicine and other various areas of technics in particular in compounds for preventive prolonged antiseptic treatment of premises with long stay of humans, for treatment of surfaces of constructional units including medical purposes and for synthesis of compounds which are biocompatible with tissue of an alive organism and preferably intended for external use at treatment of skin diseases, not healing wounds, trophic ulcers, burns, dermatosis, pustular diseases of a skin, inflammatory infiltrates.

### Prior Art

Use of the compositions containing metals such as Ag, Au, Pt, Pd, Cu, and Zn (see H. E. Morton, Pseudomonas in Disinfection, Sterilisation and Preservation, ed. S. S. Block, Lea and Febider 1977 and N. Grier, Silver and its Compounds in Disinfection, Sterilisation and Preservation, ed. S.S. Block, Lea and Febider, 1977) is widely known in practice of manufacture of fungicides and bactericides. It is also known that particles of substance having a size in the range of 1-100 nanometers change their chemical, physical and biological properties, which parameters have the important applied value. Even so significant attention has been recently paid to use of ultradisperse colloidal systems of biocidal preparations on the basis of metallic components, preferably silver, which are relevant to the most effective antimicrobic means (see Blagitko E. M., etc. <<Silver in medicine>>, Novosibirsk: “Science-Center”, 2004, 256 pages). In the technical specification of Russian Patent No. 2259871, a preparation possessing fungicidal and bactericidal properties received as a colloidal solution of a nanostructural composition of biocide on the basis of metals nanoparticles is described. The nanostructural composition of biocide is obtained by dissolution of metal salt and water-soluble polymer in water and/or in non-aqueous solvent. Then a reaction container with the received solution is blown through with gaseous nitrogen or argon and irradiated with radioactive radiation. In this method the reducer is a solvated electron generated by ionizing radiation in the solution. As salt of metal it is possible to apply a salt of at least one metal chosen from silver, copper, nickel, palladium or platinum. It is preferable to apply a salt of silver, for example nitrate, perchlorate, sulfate or acetate. As polymer polyvinylpirrolidone, copolymers of 1-vinylpirrolidone with acrylic or vinylacetic acids with styrene or with vinylic alcohol are used. As non-aqueous solvent it is possible to use methanol, ethanol, isopropyl alcohol or ethylene glycol. If emulsion is obtained surface-active substance is entered in the reaction container in addition. Obtained nanocomposite of biocide on the basis of metal-polymer is used as antibacterial, sterilizer or deodorizing means.

However the known method of obtaining a biocide is rather difficult and expensive as synthesis is carried out in an atmosphere of inert gas and with the use of a source of ionizing radiations for the purpose of preventing collateral reactions.

In the technical specification of Russian Patent No. 2088234.1997, the water-soluble bactericidal composition which contains in the structure nanoclusters of zero-valent metallic silver with the sizes 2-4 nanometers and poly-N-vynilchlorridone-2 is suggested. In the declared method poly-N-vynilchlorridone-2 acts not only as the stabilizer of colloidal silver but also as the reagent participating in restoration due to its end aldehydic groups. Thus ionic silver is restored up to molecular state by action of ethanol on the ions of silver coordinated with poly-N-vynilchlorridone-2. In absence of the last component, nitrate of silver does not react with ethanol. The compound is easily dissolved in water with formation of a colloidal solution and can be used for the manufacture of preparations for medicine and veterinary field. The preparation is characterized by the lowered toxicity and allergenicity.

However the method of obtaining this preparation is laborious and requires big power inputs as the technology of manufacturing provides for dispersion drying equipments; has also restriction of the raw-material base. Synthetic polymer increases cost of the preparation.

From the invention of Russian Patent No. 227866 it is also known to add a water solution of silver salts with contents from 0.0011 up to 0.40 g (from 0.007 up to 2 mmole) to a water solution of arabinogalactan at intensive randomization. Further it is kept at room temperature for 30-90 minutes. After that, 30% ammonium or sodium hydroxide is added up to pH 10-11. The obtained mixes are kept at a temperature of 20-90° C. for 5-60 minutes. The solution is filtered and target products are isolated by decantation of a filtrate in ethanol. The deposit is filtered and dried in vacuum. The contents of silver in the obtained compounds is determined with the method of the atomic-absorption analysis and varies within 3,3-19,9% depending on the conditions of reaction. Silver is in a zero-valent condition according to the data of the roentgen-diffractional analysis. Silver derivatives are generated as nanosized particles of 10-30 nanometers. These particles are water-soluble and can be isolated in a solid state. Silver derivatives of arabinogalactan possess antimicrobic properties and have a wide spectrum of uses. For example, derivatives with the various contents of silver can be used in medicine as antiseptic means of external use, as an alternative medical product to antibiotics and also as components of bactericidal coatings.

However use of the stabilizer, i.e. natural polysaccharide of arabinogalactan as a reducer of silver ions up to a zero-valent condition and also simultaneously as the reaction dispersive environment increases costs of preparation.

Thus, above mentioned technical solutions for obtaining preparations possessing bactericidal properties on a basis of nanostructural compositions of biocides are characterized by labour-intensiveness and at the same time by rather low stability of their liquid dispersions, owing to washing away or complex formation of free ions of silver in a solution.

The technical solution under Russian Patent No. 2330673 is the most close to the present invention. In this patent the nanostructural composition of biocide possessing fungicidal and bactericidal properties is disclosed.

According to the known technical solution the composition of biocide as nanoparticles of a bentonite powder intercalated by ions of Ag⁺ or /and of Cu²⁺ which are obtained with the process of modification of bentonite semi-finished products by 10-20% solutions of inorganic salts of silver nitrate or copper sulfate is disclosed. Bentonite semi-finished products are preliminarly enriched with cations of Na⁺ by their treatment with water solution of inorganic salts of sodium bentonite in Na⁺ form with subsequent their cleaning from acid anions after their enrichment, and from salts of sodium after the process of intercalation.

According to the known technical solution the nanostructural composition of biocide contains a basis of polar solvents.

The known nanostructural composition of biocide as nanoparticles of the bentonite powder intercalated by ions of Ag⁺ or /and of Cu²⁺ is obtained from mineral and ecologically safe components. They are biologically compatible with tissues of living organisms. The nanostructural compositions also can be used as additives for manufacturing dry building mixes, in medicine and veterinary science for antimicrobic treatment of the injured zones of tissues of living organisms, in structure of various ointment bases or of gels capable of absorbing microbic and tissues toxins.

The known nanostructural compositions of biocide can be used as a preparation, for example, for antimicrobic and fungicidal treatment of surfaces of various constructional products, for treatment of textile products and also in medicine and veterinary science for treatment of the injured zones of tissues of living organisms and in structure of preparations capable of absorbing microbic and tissues toxins.

It follows from the used technical solution that use of nanostructural compositions of biocide as a mix of nanoparticles of a bentonite powder intercalated by ions Ag⁺ and ions Cu²⁺ is the most reasonable economically. Thus, obtained biocide forms an effective synergetic composition with bactericidal and fungicidal properties.

It also follows from the applied technical solution, that bactericidal and fungicidal activity of prolonged action of biocide at treatment of surfaces of constructional products is most effective in the presence of a liquid environment, as polar solvents in the structure of the biocide. The liquid environment is safe ecologically and toxicologically. The presence of the liquid environment in a composition of biocide improves the process of its distribution on the treated surfaces, providing the maximal microbiological efficiency that is desirable at industrial application.

However compositions of biocide on the basis of a mix of nanoparticles of a bentonite powder intercalated by ions Ag⁺ and ions Cu²⁺ do not possess universality owing to possible allergenicity of tissues of living organisms at external use, in particular at treatment of never-healing wounds, trophic ulcers, burns, dermatosis, pustular diseases of a skin of patients with diabetes.

Use of nanoparticles of a bentonite powder intercalated by ions Cu²⁺ can lead to formation of electrochemical corrosion in technical means and preparations intended for treatment of surfaces of constructional products, for example, made of such metals as iron and aluminium. It also can lead to biocorrosion at its use in the products protecting wood building materials from affection by fungus, for example, telephone columns, fencings, wooden floors, braided products, windows and doors, plywood, pressed wood slabs, wafer slabs, wood-shaving slabs, joiner's products, bridges or the wooden products usually used in construction of residential buildings and other constructions.

Specifically in the applied technical solution the wide range of dispersion of nanoparticles of a bentonite powder is technologically inefficient at their compounding into liquid basis owing to possible agglomeration of nanoparticles. It reduces reliability of bactericidal and fungicidal properties of the applied composition of biocide concerning various steady forms of microorganisms and colonies of mycelial fungus.

### SUMMARY OF THE INVENTION

Technical result of the invention is the creation of nanostructural compositions of biocide. Thus the biocide is composed by a mix of nanoparticles of a bentonite powder intercalated by ions of metals, in a given weight ratio. This mix forms an inexpensive low toxic synergetic composition with effective bactericidal and fungicidal activity of prolonged action.

Technical result of the invention is the creation of the profitable nanostructural compositions of biocide possessing prolonged highly effective fungicidal and bactericidal properties for obtaining preparations intended for treatment of surfaces of constructional products without dependence from the physical-mechanical properties of these materials.

Technical result of the invention is the creation of profitable nanostructural compositions of biocide for obtaining preparations possessing prolonged, highly effective fungicidal properties concerning various steady forms of colonies of mycelial fungus.

### DETAILED DESCRIPTION OF THE INVENTION

For the solution of the described technical problem a nanostructural composition of a biocide formed of nanoparticles of a bentonite powder, intercalated by ions of Ag⁺ or/and by ions of Cu²⁺ was suggested. These nanoparticles are obtained with a process of modification of bentonite semi-finished products by 10-20% solutions of inorganic salts of silver nitrate or copper sulfate. Bentonite semi-finished products are preliminarly enriched with cations of Na⁺ by treatment with a water solution of inorganic salt, of a sodium bentonite in Na⁺ form, with subsequent its cleaning from acid anions, after enrichment, and from salts of sodium after the process of intercalation. The composition of the present invention is different in that nanoparticles of bentonite powder intercalated by ions of Zn²⁺ are additionally entered in the above said composition. These nanoparticles are obtained by treatment with 10-20% solutions of inorganic salts preferably of chloride zinc (ZnCl₂) or sulfate zinc (ZnSO₄), after the process of modification by enrichment with cations of Na⁺ of said bentonite semi-finished products, with subsequent their cleaning from sodium salts and dispersion.

The new composition has the following ratio of components (weight parts):

nanoparticles intercalated by ions of Ag⁺: nanoparticles intercalated by ions of Zn²⁺ as 1:(0,2 −0,8);

or

nanoparticles intercalated by ions of Ag+: nanoparticles intercalated by ions of Zn²⁺: nanoparticles intercalated by ions of Cu²⁺ as 1:(0,2 −0,8):(0,2−0,5);

or

nanoparticles intercalated by ions of Zn²⁺: nanoparticles intercalated by ions of Cu²⁺ as 1: (0,2 −0,5),

and dispersion of nanoparticles of bentonite powders of no more than 70 nm.

According to the invention the composition of biocide may contain a liquid basis of polar solvents.

According to the invention, solutions of the named inorganic salts of silver, copper and zinc are used to modify of semi-finished products of bentonite enriched with ions Na⁺ in the hereinafter specified weight ratio: semi-finished product:solution as 1:(10−40).

By realization of the claimed technical solution the creation of an inorganic biocide with the structure of a mix of nanoparticles of a bentonite powder intercalated by ions of said metals in the given weight ratio is ensured. It forms an inexpensive synergetic composition with highly effective bactericidal and fungicidal prolonged action on treated zones of tissues with antiallergenic effect.

By realization of the claimed technical solution, creation of the nanostructural compositions of biocide as inexpensive synergetic compositions on the basis of nanoparticles of a bentonite powder intercalated by ions of metals and of a polar solvent is provided. This composition provides highly effective bactericidal and fungicidal prolonged action on surfaces of various constructional products without dependence on physical-mechanical properties of materials, forms of microorganisms and colonies of mycelial fungus.

The obtained technical result of the invention is explained in the following way:

use of a natural mineral as bentonite in Na-form for manufacturing the preparation, whose structure is characterized by a crystal lattice with typical disposition of “packages” as level-by-level. The “packages” are represented as negatively charged aluminium-oxygen and silicon oxygen compounds where the volume of interlayer space has high sorption activity to solutions and to reaction of ionic replacement of cations of one metal with cations of other metals at presence of solutions with cations of metal-substituent in interlayer space;

performing of preliminary enrichment of bentonite in Na-form with ions Na⁺. It provides activation of bentonite owing to increase in total quantity of Na⁺ ions in its exchange capacities. They are capable of further ionic exchange at technological operations of intercalation of the ionic processes accompanying by reaction of replacement of adsorbed sodium cations on cations of other metals in exchange capacities of bentonite. As a result of reactions of ionic exchange at modification of bentonite by solutions of salts of silver nitrate (AgNO₃), copper sulfate (CuSO₄), salt of zinc (ZnCl₂), the density of Ag⁺ ions, Cu²⁺ ions, Zn²⁺ ions mainly in the interlayer space of aluminium-oxygen and silicon oxygen compounds of bentonite is raised. Processes of activation of bentonite clays due to their enrichment by ions of corresponding metals (technological treatment by salt solutions) in particular by ions of Na⁺, are used in the dehydration of cellulose masses, dehydration of paper sediments during differentiation between a liquid/firm body, in the cleaning of sewage, cleaning of water with the waste products containing inks and in the fixing of a pitch (during manufacture of paper) and also when obtaining bentonite for granulation of iron ore or for treatment of other minerals;

use of biocide dispersive environment of nanoparticles of a bentonite powder with a high specific surface in the nanostructural compositions. It provides the big area of contact to the bacterial environment and raises efficiency of antimicrobic and fungicidal influences on pathogenic microflora;

presence of nanoparticles intercalated by ions of Zn²⁺ with inhibitors of corrosion ⁻in biocide;

presence of nanoparticles of a bentonite powder intercalated by ions of Zn²⁺ in the composition biocide. Nanoparticles promote favorable antibacterial influence on tissues of homoiothermal organisms. It is widely known practice the use of Zn-containing preparations improving live ability of living organisms in medicine and in veterinary science;

use of synergetic compatible components both on the basis of mixes of bentonite powders intercalated by ions of metals and on the basis of applied liquid environment in the nanostructural compositions of biocide. They are ecologically safe to various work surfaces;

decrease in costs of obtaining a biocide due to the use of a high dispersion, synergistically compatible mix of nanoparticles of bentonite powders in its composition;

At the analysis of expert technics, it was not revealed a technical solution with a set of attributes corresponding to the technical solution according to the invention and able to realize the above described result of prolonged action of bactericidal (antimicrobic) and fungicidal efficiency on work surfaces of various constructional products and tissues of homoiothermal organisms.

The presented analysis of the state of the art testifies the conformity of the declared technical solution to criteria ornovelty” and “inventive level”.

The technical solution according to the invention can be realized industrially for obtaining the preparations intended, for example, for antimicrobic treatment of wound, burn, ulcer zones of skin integuments, for treatment of mucous surfaces of the oral cavity, for preventive and for prolonged antimicrobic and fungicidal treatment of surfaces of the constructional products made of various materials.

The essence of the invention is explained in the following way:

tables 1 and 2 expose the results on bactericidal and fungicidal efficiency of nanostructural compositions of biocide according to the invention;

recommendations concerning the choice of source of raw materials for manufacturing of the nanostructural compositions of biocide possessing fungicidal and bactericidal properties.

For obtaining of the nanostructural compositions of biocide possessing fungicidal and bactericidal properties, finished medical and laboratory equipments, commodity products and also known technological processes are used, in particular:

bentonite (montmorillonite) in Na-forms, for example, Sariguh deposit (Armenia) with alkaline bentonites in which contents of montmorillonite (bentonite in Na-forms) is 75-85 mass %. That is the most preferable to realization of the technological process of obtaining a biocide;

silver nitrate (AgNO₃); copper sulfate (CuSO₄); zinc chloride (ZnCl₂) or zinc sulfate, sodium chloride (NaCl);

deionizided water; alcohol, preferably isopropyl alcohol. The specified solvents accordingly water and alcohols concern the class of polar solvents.

the nanostructural composition of biocide possessing fungicidal and bactericidal properties (see Russian Patent No. 2330673, with a priority date of 22.11.2006, Patent holder—Joint-Stock Company <<Institute of Applied Nanotechnology>>). According to it mineralogical raw material (bentonite in Na-forms) is activated (enriched) by ions of Na⁺, by treating it with 3-10% water solution of sodium chloride, subsequent washing and filtering of the obtained semi-finished product for removal of acid anions. Then the obtained semi-finished product is modified by 10-20% solution of inorganic salts of metal such as silver nitrate (Ag NO₃) or copper sulfate—Modified bentonite is kept to mature in the specified salt solutions and then modified bentonite is cleaned from salts of sodium by washing and filtration and, after drying, the obtained preparation is reduced to powder. Thus treatment of an inorganic mineral by the named solutions is made at a ratio—weight parts of bentonite:solution as 1:(10−40).

Obtaining of the nanostructural composition of biocide, possessing antimicrobic prolonged action on a colony of bacteriological impurity and organisms, is provided at realization of the applied invention on the basis of use of the components mentioned above, of the known technological processes and with the specified weight ratio of components.

These industrial applications are typical:

for treatment of surfaces of constructional products without dependence on physical-mechanical properties of their materials;

for treatment of various infected wounds including not healing for a long time and not reacting to treatment with known means.

Nanostructural compositions of biocides obtained according to the invention are not toxic, do not cause an allergy, have no contra-indications and possess high antiedematous, sorption, ion-exchanging and antiinflammatory properties.

Realization of the invention with change of structure of the used components and of the specified weight ratio, will lead to worsening of properties of the provided biocide compositions or to increase in cost of process for their obtainment.

### Experimental Part

Realization of the invention is explained by the following steps and concrete examples of its implementation:

1st step—manufacturing of semi-finished products of bentonite preliminarly enriched with cations of Na⁺. According to it, semi-finished products are obtained with the technological process of the Russian Patent No. 2330673:

Bentonite (montmorillonite) in Na-form in amount of 5 g, is coated preferably with 5% water solution of NaCl and kept in the given solution. Thus additional enrichment of bentonite by ions of sodium is carried out. Then the obtained compound is washed for removal of chlorine anions, subsequently filtered through the filter <<a white tape>> and dried.

2nd step—obtaining of nanoparticles of a bentonite powder intercalated by ions of metals, without containing salts of sodium.

Nanoparticles of the bentonite powders without salts of sodium from semi-finished products of bentonite made at 1st step, are obtained according to the following examples:

### EXAMPLE 1

A semifinished product was cleaned from acid anions, dried up and modified by 10-20% water solution of silver nitrate (at red illumination). It was preferable to apply 15% water solution of silver nitrate. The process of modification was carried out at its keeping in the specified solution and at a temperature corresponding to its solubility in water. The obtained modified semi-finished product was repeatedly washed out for removal of sodium salts; filtered and dried preferably at a temperature higher than 200° C. and no more than 800° C. The consumption of water solutions for the treatment of 5 g semi-finished product was of bentonite : water solution as 1 : 20. After drying, the product was reduced to dispersed powder. A bentonite powder without salts of sodium and intercalated by ions of Ag⁺ was obtained. A useful yield of the product was 4.8 g

### EXAMPLE 2

The same materials and technological method as in the example 1 were used, but modification of bentonite enriched by ions of sodium, was carried out with use of 15% water solution of copper sulfate. A bentonite powder without salts of sodium and intercalated by ions of Cu²⁺ was obtained. A useful yield of the product was 4.8 g

Nanostructural compositions of biocides of Examples 1 and 2 were obtained according to the known process protracted by the Russian patent No. 2330673.

Nanoparticles of a bentonite powder intercalated by ions of Zn²⁺ to be inserted in the nanostructural compositions of biocide of the present invention, were obtained according to following Example 3.

### EXAMPLE 3

The same materials and technological methods as in the Example 1 were used, but modification of a semi-finished product of bentonite enriched by ions of sodium was carried out according to the present invention. For these purposes it was used 10-20%, preferably 15%, water solution of chloride zinc (ZnCl₂) (the most accessible chemical preparation). In result, after repeated washing for removal of sodium salts, filtering, drying and subsequent reducing to dispersed powder, a bentonite powder without salts of sodium and intercalated by ions of Zn²⁺ was obtained. A useful yield of the product was 4.8 g

The consumption of the salt of water solution for the treatment of 5 g semi-finished product was: bentonite:water solution as 1:20.

Process of dispersing powders of the invention up to the specified dispersion of nanoparticles is carried out in all Examples as follows:

The obtained products after intercalation (modification) by ions of metals, their cleaning from salts of sodium and drying, are slurried (intensively mixed) in plenty of water and are allowed to settle during some time. The decanted product is slurried in additional portion of water the deposit is slurried, settled and decanted again. This process is carried out ,repeatedly. By filtration from decanted liquids a nanodispersion product is isolated. Then it is dried and grinded in planetary mills. A plenty of deionized water is used in such a way of obtaining nanopowders. The process is rather long.

For reducing the time of processing to nanoparticles, the named products of Examples 1-3 were compounded in deionized water at the following ratio of weight parts: a product (Examples 1-3): solvent as 1:10. Then it was carried out formation of a dispersion of nanoparticles of the bentonite powder up to the dimension of them of no more than 70 nm, with use of ultrasonic dispersant.

Ultrasonic dispersant are widely used in various industries (chemical, pharmaceuticals, food, etc.). As a source of ultrasound either hydrodynamical radiators or radiators on the basis of electro-mechanically active materials are used, for example, magnetostrictive converters. Use of ultrasonic dispersant will considerably speed up the process of structuring bentonite powders up to the specified value of dispersion.

In a case the process was carried out with use of dispersant Bandelin Sonoplus HD2070 at capacity 40 Watt for 10-20 minutes. Obtained colloidal systems were deposited on a sublayer and after evaporation of water were scanned by a microscope.

The control of dimension of obtained dispersion of bentonite powders was made with use of an electronic microscope. As a result of the carried out technological methods, dispersion of nanoparticles to less than 70 nanometers was obtained, with the following distribution: dispersion of 30% from total structural product was of 5-20 nanometers, the rest was less than 70 nm.

Dispersions of bentonite powders, intercalated by ions of the named metals, with dimension of nanoparticles less than 70 nm were used for the preparation of mixes of the nanostructural compositions of biocide of the invention:

### EXAMPLE 4

Obtained nanoparticles of the bentonite powders intercalated by ions of Ag⁺ and Zn²⁺ (Examples 1 and 3) were mixed at a ratio of their weight parts of:

the product of Example 1 : the product of Example 3 as 1:0, 5.

Obtained mix of nanoparticles of bentonite powders was compounded into polar solvent preferably into deionized water at the following ratio:

Mix of bentonite powders of Example 4:polar solvent as 1:20.

5% liquid solution of the composition of biocide is obtained.

### EXAMPLE 5

Obtained nanoparticles of bentonite powders intercalated by ions of Ag⁺, Zn²⁺ and Cu²⁺ (Examples 1, 2 and 3) were mixed at a ratio of their weight parts of:

the product of Examplel : the product of Example 3: the product of Example 2 as 1:0, 5 : 0,3.

Obtained mix of nanoparticles of bentonite was compounded into polar solvent preferably into deionized water, at the following ratio:

Mix of bentonite powders of Example 5: polar solvent as 1:20.

5% liquid solution of the composition of biocide is obtained.

### EXAMPLE 6

A mix of nanoparticles of bentonite powders as used in Example 5 was compounded in a polar solvent as 40% of a hydro-alcoholic solution, at the following ratio of weight parts:

the product of Example 5: solvent as 1:20

5% liquid solution of the composition of biocide is obtained.

### EXAMPLE 7

Nanoparticles of bentonite powders intercalated by ions of Zn²⁺ and Cu²⁺ according to Examples 2 and 3, were mixed at a ratio of weight parts of:

the product of Example 3: the product of Example 2 as 1:0, 5

The mix of nanoparticles of bentonite powders was compounded into deionized water at the following ratio:

mix of bentonite powders of Example 7: polar solvent as 1:20

5% liquid solution of the composition of biocide is obtained.

Control compositions of biocides have been prepared additionally for carrying out comparative tests of Examples 8 - 10:

### EXAMPLE 8

The product of Example 1: polar solvent (deionized water) as 1:20

### EXAMPLE 9

The product of Example 2: polar solvent (deionized water) as 1:20

In all examples 1-9 used nanoparticles of bentonite powders had dispersion of no more than 70 nm.

### EXAMPLE 10

The product of Example 1: polar solvent (deionized water) as 1:20,

dispersion was no more than 100 nm (dispersion of 30% of nanoparticles was no more than 30 nm and dispersion of 70% was 100 nm).

Biocidal properties of the preparations obtained in accordance to Examples 1-10 were estimated on bactericidal and fungicidal activity of tested samples.

Estimation of bactericidal (antimicrobic) activity of nanostructural compositions of tested samples was carried out with use of an integrated disk-diffusion method. (the Directions on medical microbiology. General and sanitary microbiology. Under edition of A. S. Labinskaya, E. G. Volina. Moscow, BINOM, 2008, pages 342-352.)

The specified method is based on diffusion of a tested antimicrobic preparation in a dense nutrient medium.

The method consisted in unitary treatment of standard disks in diameter of 5 mm by tested samples.

Disks were placed on the surface of a dense nutrient medium (a tripkazo-soy agar (TSA) manufactured by bioMerieux, France) preliminarly inoculated by one of test-microorganisms.

Petri's cup with cultures of test-microorganisms and the disks treated with water solutions of samples were placed in thermostat for 24-48 hours at a temperature of 37° C.

After the expiration of the specified term, results of the research were determined by measurement of diameter of a zone of a growth delay of test-microorganisms in mm around of the disks. Each research was repeated three times.

24-hour cultures of bacteria of Staphylococcus aureus, Pseudomonas aeruginosa and spores of bacteria of Bacillus cereus were used as test-microorganisms.

For preparation of a suspension of test-culture of spore—generating bacteria the daily culture was used, grown up on a dense nutrient medium (a tripkazo-soy agar) at a temperature of 37° C. Then suspension of culture in a physiological solution was prepared for stimulation of spore—generating at bacteria of sort Bacillus. It was dispersed on a surface of a potato agar poured in sterile Petri's cup in volume of 0.2-0.5 ml on a cup. Incubation period was in thermostat for 48 hours at a temperature of 37° C. After incubation Petri's cup with plating microorganisms were pulled out from thermostat and kept at room temperature (20-22° C.) in the presence of a natural light source for 5 days.

Further control was carried out on test-cultures of bacteria which have been grown up on a potato agar for 7 days under various conditions of incubation. This research was directed to reveal spore-generating bacteria of sort Bacillus. For this purpose a preparation of test-culture of bacteria was prepared, it was painted over by the method of Shaffer-Fulton and examined under a microscope. If about 90-95° A, spores of bacteria are visible at survey of the preparation under a microscope, the preparation of the spore-generating test-culture could be used for preparation of a suspension. Otherwise the test-culture of bacteria needs further incubation.

A suspension of each test-culture of bacteria was prepared in a sterile physiological solution using the reference glass standard of turbidity on 10 units. It corresponds to an amount of microbic cells of 1 billion/ml. Then the concentration of a suspension of test-microorganisms equal to 10⁶ cells in 1 ml by series of consecutive cultivations in a sterile physiological solution was obtained. The suspension of each kind of bacteria with the specified concentration was deposited on the surface of a nutrient medium. On the obtained bacterial area the disks impregnated with the researched preparations according to examples 4-10, were imposed.

Bacteria of Staphylococcus aureus are chosen as one of the most resistant representatives of the gram-positive microflora of the human being. Besides, they are one of the basic activators of hospital infections and also activators of pustular infections of a skin, furuncles, abscesses and other complications.

Bacteria of Pseudomonas aeruginosa (strain ATCC No. 10145) are chosen as one of the most resistant representatives of the gram-negative flora possessing high stability to physical and chemical factors. As a rule, they show resistance to many medicinal and to disinfectants. Besides, bacteria of the given kind are known as activators of infectious complications of bum wounds, bacteremias, septicemias with a fatal outcome and other complications of infectious aetiology.

Bacteria of Bacillus cereus (strain No. 8035 NCTC) are chosen as representatives of spore-generating microorganisms which spores are the steadiest to be influenced by adverse factors of an environment including action of disinfectants. Both activators of infectious diseases and activators of biocorrosion of constructional materials are available among them. As a rule, spores of bacteria of Bacillus are used for tests of work of autoclaves, dry-heating cases and disinfectants.

Results of the carried out researches are submitted in the Table 1. Estimation of fungicidal (antifungicidal) activity of tested samples (Examples 4-10) was carried out with an integrated disk-diffusion method as mentioned above.

The method consists in unitary treatment by tested samples of standard disks in diameter of 5 mm.

Disks were placed on the surface of a dense nutrient medium (Czapek Dox agar manufactured by Himedia, India) preliminarly inoculated with one of test-microorganisms.

Petri's cup with cultures of test-microorganisms and the disks processed with the mentioned samples were placed in thermostat for 5-7, 24 hours at a temperature of 28° C.

After the expiration of the specified term the result of researches were determined by measurement of the diameter of a zone of a growth delay of tests-microorganisms in mm around the disks.

A number of samples of each sort was taken in view of carrying out an estimation of each parameter at least on 3 samples.

As test-microorganisms cultures of fungus of Aspergillus sydown (pieces 9-6), Aspergillus niger (pieces 4-3-11), Cladosporium cladosporioides pieces (2-3), Penicillium expansum (pieces 4-3-3), Ulocladium botrytis (pieces 15-10) were used. These strains have been isolated from an inhabitancy of the International space station and possessed stability to the influence of adverse factors of an environment including the action of disinfectants. As a rule fungus of Aspergillus niger are used for tests of disinfectants.

For standardization of test-cultures, strains were grown in Petri's cup with the medium of Capek. Their specific identity was confirmed on the basis of the analysis of their cultural and morphological properties. Then they were placed on the oblique agar (Capek's medium) poured out in big test tubes (diameter of 20-22 mm). Cultures were grown in thermostat at a temperature of 28° C. for 10-14 days. Strains thus obtained were kept in a refrigerator at a temperature of +4° C. and, as required they were placed out and used for preparation of a suspension. For preparation of a suspension of fungus strains the test-cultures of fungus were used. They had been grown up in Capek's medium at 28° C. at ages from 14 till 28 days beginning from the moment of plating.

Suspension of strains in concentration of 1 million/ml was prepared separately for each kind of test-cultures of fungus. For this purpose strains of fungus from a test tube with pure culture were transferred in a flask (test tube) containing 15±5 ml of a sterile physiological solution. Transfer of strains from test tubes in a flask (test tube) was made by the method of holding of strains by a bacteriological loop.

At intake of strains from a test tube a nutrient medium was not touched by a loop. Determination of amount of strains in suspension was carried out by a method of calculation with use of accounting chamber of Gorjaeva.

Suspension of every kind of fungus with the specified concentration was placed on the surface of a nutrient medium (a lawn of culture). On the obtained lawn of fungus the disks impregnated by researched samples (Examples 4-10) were placed.

Results of the carried out researches are submitted in the Table 2.

From the analysis of the Tables 1 and 2 it results what follows:

Water and hydroalcoholic solutions of nanostructural compositions of biocide (examples 4-10) possess antimicrobic activity in respect to representatives of gram-positive, gram-negative and spore-generating flora (Table 1).

It follows from the Table 1 that testing samples (Sample 8) containing nanoparticles of bentonite intercalated by ions of silver (Ag⁺), zones of a growth inhibition around of the disks with bacteria of S. aureus was 20 mm, with bacteria of P. aeruginosa was 18 mm and with spore-generating bacteria of B. cereus was 11 mm. These data testify the efficiency of bactericidal activity of silver preparations. It is known for the given kind of metal possessing a wide spectrum of antimicrobic activity. At the same time it is known that costs for manufacturing the given product are considerably high and thus inexpedient.

As a result of tests it is also determined that tested samples according to the invention (Samples 4-7), containing a mix of nanoparticles of bentonite with ions of metals according to the invention, insignificantly differ from the product of the Sample 8 for the bactericidal properties of prolonged action. And in comparison with it, costs for obtaining samples 4-7 are lower.

As a result of the tests it is also determined that significant growth of Bacillus (Bacillus cereus strain No. 8035 NCTC) took place by using a composition of biocide on the basis of nanoparticles of a bentonite powder intercalated by ions Cu²⁺ (Sample 9). That testifies the presence of biocorrosion processes.

It is also determined that the efficiency of bactericidal properties of preparations according to the invention (Sample 10), considerably decreases in the presence of product with significant part of nanoparticles of bentonite powders with dispersion of more than 70 nm.

The best fungicidal properties were shown by preparations of Examples 4-8.

Fungistatic and fungicidal properties were determined in these preparations. They showed a different degree of influence of their activity on various kinds of fungus (Table 2). The most sensitive to the specified tested preparations were dark-colored fungus showed and the most resistant funguses were aspergillus. It follows from researches on estimation of fungicidal (antifungicidal) activity, that tested samples according to the invention (Samples 4-7) containing a mix of nanoparticles of bentonite with ions of metals according to the invention, insignificantly differ from the product by the Sample 8 (control example) in respect to the fungistatic and fungicidal properties of prolonged action. And in comparison with it, costs for obtaining Samples No. 4-7 are lower.

It is also determined that efficiency of fungicidal properties of preparations (Sample 10) considerably decreases at dispersion of nanoparticles of bentonite powders more than 70 nm.

Thus, the carried out researches as a whole confirm high efficiency of bactericidal and fungicidal properties of prolonged action of nanostructural compositions of biocide to various colonies of microorganisms under the applied invention, what testifies the expediency of use of the invention for the following purposes:

for antimicrobic treatment of wounded, burn, ulcer zones of integuments, for treatment of mucous surfaces of the oral cavity without intoxication of treated zones;

for treatment of surfaces of constructional products without dependence from properties of the materials used for their manufacturing.

TABLE 1
Antibacterial activity of test samples

| | | Type | | |
|---|---|---|---|---|
| | | Staphylococcus | Pseudomonas | Bacillus |
| | | aureus | aeruginosa | cereus |
| | | Diameter of a zone of a growth inhibition | | |
| | | of tests-microorganisms around of standard | | |
| | | disks processed by tests samples in mm. | | |
| No. | Tested samples | Disk diameter - 5 mm. | | |
| 1 | Sample 4 | 18 | 17 | 10 |
| | (Ag⁺ + Zn²⁺ + water) | | | |
| 2 | Sample 5 | 19 | 17 | 10 |
| | (Ag⁺ + Cu²⁺ +Zn²⁺ + | | | |
| | water) | | | |
| 3 | Sample 6 | | | |
| | (Ag⁺ + Cu²⁺ +Zn²⁺ + | 20 | 18 | 11 |
| | hydroalcoholic basis) | | | |
| 4 | Sample 7 | 17 | 16 | 9 |
| | (Cu²⁺ + Zn²⁺ + water) | | | |
| 5 | Sample 8 | | | |
| | (Ag⁺ + water) | 20 | 18 | 11 |
| | reference sample | | | |
| 6 | Sample 9 | | | |
| | (Cu²⁺ + water) | 14 | 11 | 6 |
| | reference sample | | | |
| 7 | Sample 10 | | | |
| | (Ag⁺ + Cu²⁺ + Zn²⁺ + | 12 | 10 | 7 |
| | water) | | | |

TABLE 2
Fungicidal properties of test samples

| | | Type of tests-culture | | | | |
|---|---|---|---|---|---|---|
| | | Aspergillus | Aspergillus | Cladosporium | Penicillium | Ulocladium |
| | | sydowii | niger | cladosporioides | expansum | botrytis |
| | | Diameter of a zone of a growth inhibition of tests- | | | | |
| | | microorganisms around of standard disks processed by | | | | |
| No. | Tested samples | tests samples in mm. Disk diameter - 5 mm. | | | | |
| 1 | Sample 4 | 0 | 0 | 4 | 2 | 2 |
| | (Ag⁺ + Zn²⁺ + water) | | | | | |
| 2 | Sample 5 | 0 | 1 | 5 | 2 | 2 |
| | (Ag⁺ + Cu²⁺ + Zn²⁺ + water) | | | | | |
| 3 | Sample 6 | 0 | 1 | 5 | 2 | 2 |
| | (Ag⁺ + Cu²⁺ + Zn²⁺ + | | | | | |
| | hydroalcoholic basis) | | | | | |
| 4 | Sample 7 | 0 | 0 | 4 | 2 | 2 |
| | (Cu²⁺ + Zn²⁺ + water) | | | | | |
| 5 | Sample 8 | 0 | 0 | 5 | 2 | 2 |
| | (Ag⁺ + water) | | | | | |
| 6 | Sample 9 | 0 | — | 2 | 0 | 1 |
| | (Cu²⁺ + water) | | | | | |
| 7 | Sample 10 | 0 | 0 | 2 | 1 | 1 |
| | (Ag⁺ + Cu²⁺ + Zn²⁺ + water) | | | | | |

(—) - in 5 days growth if the test-culture of funguses (Aspergillus niger) on the disk is exposed;
(0) - in 5 days the test-culture does not grow on the disk.

1. Nanostructural biocide composition, with fungicidal and bactericidal activity, consisting of nanoparticles of bentonite powders intercalated by Zn2+ ions and by at least one ion selected between Ag+ and Cu2+.

2. Nanostructural biocide composition according to 5 claim 1 where the nanoparticles of bentonite powders intercalated with the metal ions, contain ions of Ag+, Zn2+, Cu2+ in the following ratio by weight among them:
nanoparticles intercalated by ions of Ag+: nanoparticles intercalated by ions of Zn2+:
nanoparticles intercalated by ions of Cu2+ as 1:(0,2−0,8):(0,2−0,5)

3. Nanostructural biocide composition according to claim 1 where the nanoparticles of bentonite powders, intercalated with the metal ions, contain only ions of Ag+ and Zn2+, in the following ratio by weight between them: nanoparticles intercalated by ions of Ag+: nanoparticles intercalated by ions of Zn2+ as 1:(0,2−0,8)

4. Nanostructural biocide composition according to claim 1, where the nanoparticles of bentonite powders, intercalated with the metal ions, contains only ions of Zn2+ and Cu2+ in the following ratio by weight between them:
nanoparticles intercalated by ions of Zn2+: nanoparticles intercalated by ions of Cu2+ as 1:(0,2−0,5).

5. Nanostructural biocide composition according to claim 1 where the dispersed nanoparticles of bentonite powders have mainly dimensions up to 70 nm.

6. Nanostructural biocide composition according to claim 1, in liquid form, consisting of the nanoparticles of bentonite powders intercalated by Zn2+ ions and by at least one ion selected between Ag+ and Cu2+, compounded with a polar solvent in a ratio nanoparticles of bentonite: polar solvent as 1:20.

7. Nanostructural biocide composition in liquid form according to claim 6, where the polar solvent is deionized water.

8. Nanostructural biocide composition in liquid form according to claim 6, where the polar solvent is a 40% hydro-alcoholic solution.

9. Process for the preparation of nanostructural biocide composition, with fungicidal and bactericidal activity, consisting of nanoparticles of bentonite powders intercalated by Zn²+ ions and by at least one ion selected between Ag+ and Cu2+, comprising all or in part the following stages
a) enrichment of a bentonite in Na-form with Na+ ions, by treatment with a 3-10% water solution of sodium chloride, subsequent washing up to removal of acid anions and drying.
b) treatment of the product obtained in stage (a) with a 10-20% water solution of an Ag salt, preferably silver nitrate, at a temperature corresponding to its solubility in water, followed by washing until removal of sodium salts and by filtering and drying;
c) treatment of the product obtained in stage (a) with a 10-20% water solution of a Zn salt, preferably zinc chloride, at a temperature corresponding to the water solubility of the salt, followed by washing until removal of sodium salts and by filtering and drying;
d) treatment of the product obtained in stage (a) with a 10-20% water solution of a copper salt, preferably copper sulphate, at a temperature corresponding to water solubility of the salt, followed by washing until removal of sodium salts and by filtering and drying

10. Process according to claim 9 comprising all the stages (a) to (d)

11. Process according to claim 9 comprising the stages (a), (b), (c).

12. Process according to claim 9 comprising the stages (a), (c), (d).

13. Process according to anyone of claims 8, 9, 10 wherein the fractions of bentonite powder obtained at the end of the stages (b), (c), (d) are dispersed in such a way that nanoparticles of bentonite with dimensions mainly not superior to 70 nm are obtained, and then are compounded among them.

14. Process according to claim 9 wherein the fractions of bentonite powder obtained at the end of the stages (b), (c), (d) are first compounded among them and then dispersed in such a way that nanoparticles of bentonite with dimensions mainly not superior to 70 nm are obtained.

15. Process according to claim 9 wherein the dispersion of the bentonite powders, up to nanoparticles with dimensions mainly not superior to 70 nm, is performed by repeated intensive mixing in a plenty of water and successive decanting, drying and grinding in a suitable mill.

16. Process according to claim 9 wherein the dispersion of the bentonite powders, up to nanoparticles with dimensions mainly not superior to 70 nm is performed by admixing the powder with deionized water in a ratio 1:10 and then applying an ultrasonic dispersant.
